(19) United States
(12) Patent Application Publication
(10) Pub. No.: US 2022/0387632 A1
(43) Pub. Date: Dec. 8, 2022

(54) RENAL CLEARABLE FLUORESCENT CONTRAST AGENT WITH INCREASED TUMOR SPECIFICITY, AND IMAGING METHOD USING SAME

(71) Applicants: The General Hospital Corporation, Boston, MA (US); NAWOOVISION CO., LTD., Miryang-si Gyeongsangnam-do (KR)

(72) Inventors: Hak-Keun Lee, Iksan-si Jeollabuk-do (KR); Taehoon Kim, Yangsan-si Gyeongsangnam-do (KR); Hak Soo Choi, Needham, MA (US); Kai Bao, Dover, MA (US)

(21) Appl. No.: 17/618,273

(22) PCT Filed: Jun. 14, 2019
(86) PCT No.: PCT/KR2019/007172

§ 371 Date: Dec. 10, 2021

(30) Foreign Application Priority Data
Jun. 13, 2019 (KR) 1020190070047

(51) Int. Cl. A61K 49/00; A61K 47/60; A61P 35/00

(52) U.S. Cl. CPC A61K 49/0032; A61K 47/60; A61P 35/00

(57) ABSTRACT

The present invention relates to a tumor-specific fluorescence contrast agent. Specifically, the present invention relates to: a renal excretion-type fluorescence contrast agent which can exhibit a high signal-to-background ratio when optically observing tumor tissues and cells using a fluorescence imaging device for image-guided surgery; and an imaging method using the same.

### TECHNICAL FIELD

The present invention relates to a tumor-specific fluorescence contrast agent. Specifically, the present invention relates to: a renal excretion-type fluorescence contrast agent which can exhibit a high signal-to-background ratio when optically observing tumor tissues and cells using a fluorescence imaging device for image-guided surgery; and an imaging method using the same.

### BACKGROUND ART

In the treatment of a disease, it is important to detect morphological and functional changes caused by an in vivo disease at the early stage of the disease. Especially during the treatment of cancer, the site and size of tumors are crucial determinants in effective treatment design. Examples of methods known for these purposes include a biopsy by perforation and the like, and an imaging diagnostic method such as X-ray imaging, MRI, and ultrasonic imaging. Although the biopsy is effective for the final diagnosis, the biopsy simultaneously puts a heavy burden on a test subject and is not suitable for tracking changes over time within the lesion. X-ray imaging and MRI inevitably expose a test subject to radiation and electromagnetic waves. Further, as mentioned above, the imaging diagnostic method in the related art requires a long time for complicated operation, measurement and diagnosis. In addition, it is difficult to apply these methods during the operation of most of the equipment used for the purposes.

One of the imaging diagnostic methods is fluorescence imaging. Fluorescence imaging is a method of labeling a cell, tissue, or living body with a fluorescent substance that absorbs light with a specific wavelength from the outside to emit light with a longer wavelength and visualizing the cell, tissue, or living body. Fluorescent molecular imaging is characterized by emitting the most efficient image signal in a near infrared region where absorption, scattering, autofluorescence, and the like of light occurring in living tissue are minimized. This is because hemoglobin, which is present in a large amount in blood vessels, absorbs light in the visible light region, and water and fat in the living body usually absorb light in the infrared region. Therefore, when a near infrared fluorescent material is applied to optical imaging, the tissue penetration power is the best and noise is minimized.

However, although imaging technology using near infrared (NIR) fluorescence has great potential in the medical field, particularly in diagnosis and image-guided surgery, there is a major obstacle of a lack of suitable contrast agents (imaging agents). A phosphor must have excellent in vivo properties such as solubility, biodistribution and clearance, and have good optical properties in order to be clinically applied.

Since most of the phosphors have poor in vivo distribution and excretion properties, it is difficult to clinically apply the phosphors. For example, phosphors have a strong tendency to be removed through the liver, which allows fluorescence to be expressed throughout the stomach and intestine (gastrointestinal tract), which results in a very strong background signal. When the background signal becomes strong, it becomes difficult to distinguish the background signal from an area to be actually observed.

Therefore, there is a need for research and development on an improved new near infrared fluorescence contrast agent, particularly, a near infrared fluorescence contrast agent which rapidly reaches a stable equilibrium state in an intravascular or extravascular space, and whose remaining materials can be excreted out of the body by renal filtration after reaching target tissues and organs.

### DISCLOSURE

### Technical Problem

The present invention has been made in an effort to solve the problems of a fluorescence contrast agent in the related art, and an object thereof is to provide a renal excretion-type fluorescence contrast agent which can exhibit a high signal-to-background ratio when optically observing tumor tissues and cells using a fluorescence imaging device for image-guided surgery.

An object of the present invention is to provide a near infrared fluorescence contrast agent which rapidly reaches a stable equilibrium state in an intravascular or extravascular space, and can be efficiently excreted out of the body by renal filtration.

An object of the present invention is to provide a renal excretion-type fluorescence contrast agent for improving a tumor signal-to-background ratio, which allows a drug to have excellent in vivo distribution and ex vivo excretion properties by charge balancing and enables high-resolution imaging by reducing undesired non-specific binding, and an imaging method using the same.

An object of the present invention is to provide a fluorescence contrast agent for improving a tumor signal-to-background ratio, which absorbs light in the near infrared (NIR) region, emits fluorescence, and has a function of being attached only to specific cells or tissues in the living body after intravenous injection, and an imaging method using the same.

An object of the present invention is to provide a renal excretion-type fluorescence contrast agent for improving a tumor signal-to-background ratio, which can be conveniently used and usefully used for image-guided surgery by realizing a function capable of expressing fluorescence and a function of being attached to specific cells and organs with a molecule having a single chemical structure.

### Technical Solution

On the one hand, the present invention provides a renal excretion-type fluorescence contrast agent for improving a tumor signal-to-background ratio, which is:

a contrast agent for imaging tumor tissues or tumor cells,

in which the contrast agent includes a compound which emits fluorescence such that a tumor signal-to-background ratio is detected at 1.1 or higher,

the compound is a compound of Chemical Formula 1 or 2, and

the contrast agent increases renal excretion of the materials remaining in vivo after tumor targeting.

On the other hand, the present invention provides an imaging method using a fluorescence contrast agent for increasing renal excretion, the method including:

(a) attaching a phosphor represented by a compound of Chemical Formula 1 or 2, or a contrast agent including the same to tumor tissues or cells;

(b) emitting fluorescence from the tumor tissues or cells by causing the phosphor or contrast agent exposed to an excitation light source to produce fluorescence;

(c) increasing renal excretion of the remaining materials in the body after tumor targeting by emitting fluorescence; and

(d) imaging the tumor tissues or cells by observing a fluorescence signal emitted from the tumor tissues or cells at a signal-to-background ratio of 1.1 or higher.

### Advantageous Effects

The renal excretion-type fluorescence contrast agent for improving a tumor signal-to-background ratio as described above and the imaging method using the same have effects as follows.

First, it is possible to exhibit a high signal-to-background ratio when optically observing tumor cells or tumor tissues using a new renal excretion-type fluorescence contrast agent and a fluorescence imaging device for image-guided surgery using the same.

Second, provided is a near infrared fluorescence contrast agent which rapidly reaches a stable equilibrium state in an intravascular or extravascular space, and can be efficiently excreted out of the body by renal filtration.

Third, a drug is allowed to have excellent in vivo distribution and ex vivo excretion properties by charge balancing and high-resolution imaging is enabled by reducing undesired non-specific binding.

Fourth, provided is a fluorescence contrast agent which absorbs light in the NIR region, emits fluorescence, and has a function of being attached only to specific cells or tissues in the living body after intravenous injection.

Fifth, it is possible to be conveniently used and usefully used for image-guided surgery by realizing a function capable of expressing fluorescence and a function of being attached to specific cells and organs with a molecule having a single chemical structure.

### DESCRIPTION OF DRAWINGS

FIG. 1 is a configuration view illustrating 700 nm and 800 nm NIR fluorescence properties of a renal excretion-type fluorescence contrast agent for improving a tumor signal-to-background ratio according to the present invention.

FIG. 2 is a process view illustrating a method for producing the contrast agent according to the present invention.

FIG. 3 is a process view illustrating a method for producing the contrast agent according to the present invention.

FIG. 4 is an example of image-guided surgery and measuring a tumor signal-to-background ratio using FIAT-L among real-time imaging equipment.

FIG. 5 is a set of photographs illustrating examples of fluorescence contrast agents using real-time imaging equipment (Du, the duodenum; In, the small intestine; Ki, the kidneys; Li, the liver; Lu, the lungs; Mu, muscle; Pa, the pancreas; Sp, the spleen).

FIG. 6 is a set of photographs illustrating examples of a renal excretion-type fluorescence contrast agent measured using real-time imaging equipment.

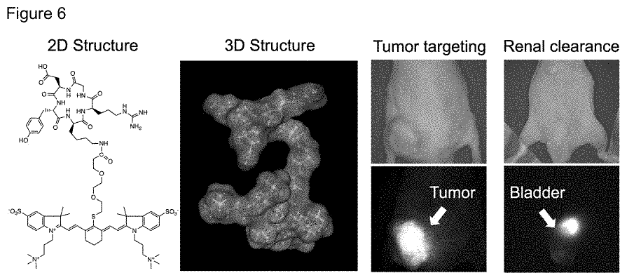

### MODES OF THE INVENTION

Hereinafter, the present invention will be described in more detail.

A renal excretion-type fluorescence contrast agent for improving a tumor signal-to-background ratio according to an exemplary embodiment of the present invention is

a contrast agent for imaging tumor tissues or tumor cells,

in which the contrast agent includes a compound which emits fluorescence such that a tumor signal-to-background ratio is detected at 1.1 or higher,

the compound is a compound of Chemical Formula 1 or 2, and

the contrast agent increases renal excretion of the materials remaining in vivo after tumor targeting.

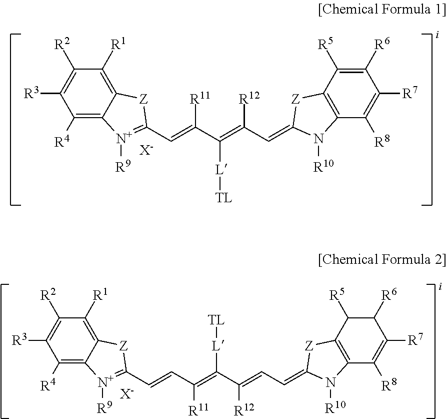

In the formulae,

R¹ to R¹² are each independently hydrogen, (Y)ₘSO₃⁻, (Y)ₘCOO⁻, (Y)ₘPO₃H⁻, (Y)ₘNH₂(CH₃)⁺, (Y)ₘNH(CH₃)₂⁺, (Y)ₘN(CH₃)₃⁺, a C₁-C₄ alkyl group, a C₁-C₄ alkoxy group, or a C₂-C₁₂ alkenyl group,

R¹¹ and R¹² may be linked to each other to form an aliphatic or aromatic ring,

Y is CH₂ or CH₂CH₂O,

m is an integer from 0 to 9,

L′ is a structure linked to CH₂, hydrogen (H), oxygen (O), nitrogen (N), sulfur (S), or selenide (Se),

TL is a targeting ligand,

X is F, Cl, Br, or I,

Z is S, Se, O, CH═CH, or C(CH₃)₂, and

i is +4, +3, +2, +1, 0, −1, −2, −3, or −4.

The compound of Chemical Formula 1 may be used when absorbing light in a region of 700 nm, and the compound of Chemical Formula 2 may be used when absorbing light in a region of 800 nm.

The near infrared (NIR) phosphors represented by Chemical Formulae 1 and 2 have a complex chemical structure including sufficient double bonds for absorbing NIR light and emitting light with a wavelength (generally a longer wavelength) different from that of the absorbed light.

A contrast agent proposed by the present invention serves to absorb light in the NIR region, emit fluorescence, and be attached only to tumor cells or tissues in vivo after being injected into the body.

Therefore, the contrast agent according to the present invention can be conveniently used and usefully used for image-guided surgery by realizing a function capable of expressing fluorescence and a function of being attached to specific cells and organs with a molecule having a single chemical structure.

The fluorescence contrast agent of the present invention has excellent optical imaging properties particularly due to its excellent behavior in vivo. More specifically, charge balancing allows a drug to have excellent in vivo distribution and ex vivo excretion properties, and reduces undesired non-specific binding. These in vivo properties enable high resolution imaging by improving a signal-to-background ratio of imaged tumor tissues or cells.

In an exemplary embodiment of the present invention, L′ is a structure linked to CH₂, hydrogen (H), oxygen (O), nitrogen (N), sulfur (S), or selenide (Se). When linked to a site called a meso carbon (center) of a polymethine backbone, the compounds are bound via L, and the form thereof is not limited.

In an exemplary embodiment of the present invention, L′ may include a PEG linker represented by a compound of the following Chemical Formula 3.

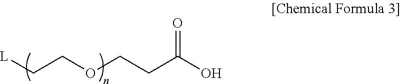

In the formula,

L is S, Se, O, or CH₂, and

n is an integer from 0 to 114.

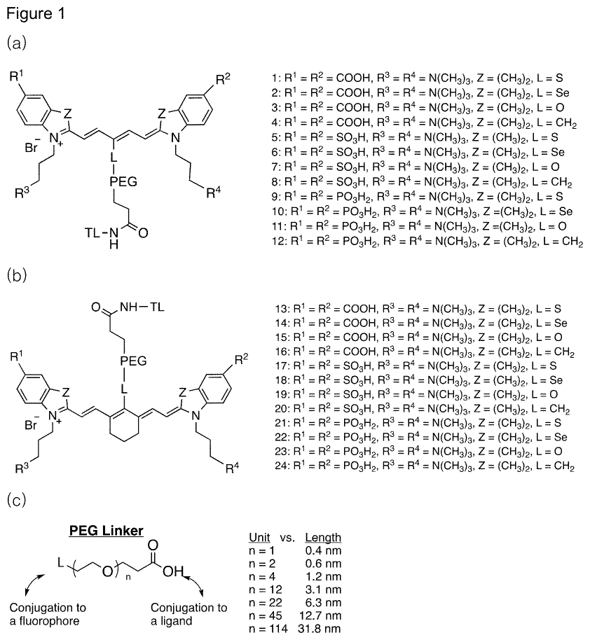

Specifically, L′ is preferably -L-PEG-(CH₂)₂CONH—, and the PEG is polyethylene glycol (see FIG. 1 (C)).

FIG. 1 is a representative chemical configuration view illustrating 700 nm (a) and 800 nm (b) fluorescence properties of a renal excretion-type fluorescence contrast agent for improving a tumor signal-to-background ratio according to the present invention.

The compound of Chemical Formula 1 or 2 included in the contrast agent according to the present invention may excrete unreacted materials remaining in the body after tumor targeting, such as fluorescence emission.

Through this, the fluorescence contrast agent according to the present invention can be applied as a renal excretion-type contrast agent.

In an exemplary embodiment of the present invention, the contrast agent may further include a compound of the following Chemical Formula 4 or 5.

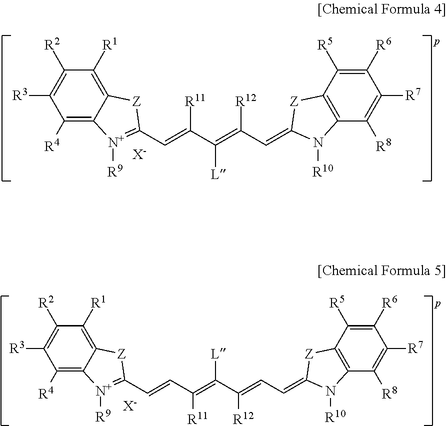

In the formulae,

R¹ to R¹² are each independently hydrogen, a C₁-C₄ alkyl group, a C₁-C₄ alkoxy group, or a C₂-C₁₂ alkenyl group,

L″ is hydrogen (H), fluorine (F), chlorine (Cl), bromine (Br), or benzene,

X is F, Cl, Br, or I,

Z is S, Se, O, CH═CH, or C(CH₃)₂, and

p is an integer from −6 to +6.

As used herein, the C₁-C₄ alkyl group refers to a monovalent straight-chained or branched hydrocarbon having 1 to 4 carbon atoms, and examples thereof include methyl, ethyl, n-propyl, i-propyl, n-butyl, and the like, but are not limited thereto.

As used herein, the C₁-C₄ alkoxy group refers to a straight-chained or branched alkoxy group having 1 to 4 carbon atoms, and includes methoxy, ethoxy, n-propaneoxy, and the like, but is not limited thereto.

As used herein, the C₂-C₁₂ alkenyl group refers to a straight-chained or branched unsaturated hydrocarbon having 2 to 12 carbon atoms, which has one or more carbon-carbon double bonds, and examples thereof include ethylenyl, propenyl, butenyl, pentenyl, and the like, but are not limited thereto.

In the C₁-C₄ alkyl group, the C₁-C₄ alkoxy group, and the C₂-C₁₂ alkenyl group, one or more hydrogens may be substituted with a C₁-C₅ alkyl group, a C₂-C₆ alkenyl group, a C₂-C₆ alkynyl group, a C₃-C₁₀ cycloalkyl group, a C₃-C₁₀ heterocycloalkyl group, C₃-C₁₀ heterocycloalkyloxy, a C₁-C₅ haloalkyl group, a C₁-C₅ alkoxy group, a C₁-C₅ thioalkoxy group, a sulfonate group, an aryl group, an acyl group, hydroxy, thiol, a halogen, amino, alkoxycarbonyl, carboxy, carbamoyl, cyano, nitro, and the like.

The imaging method according to an exemplary embodiment of the present invention includes:

(a) attaching a phosphor represented by the compound of Chemical Formula 1 or 2, or a contrast agent including the same to tumor tissues or cells;

(b) emitting fluorescence from the tumor tissues or cells by causing the phosphor or contrast agent exposed to an excitation light source to produce fluorescence;

(c) increasing renal excretion of the remaining materials in the body after tumor targeting by emitting fluorescence; and

(d) imaging the tumor tissues or cells by observing a fluorescence signal emitted from the tumor tissues or cells at a signal-to-background ratio of 1.1 or higher.

In an exemplary embodiment of the present invention, the contrast agent may be produced by:

selecting a dye whose absorption spectrum has a maximum value at 600 nm to 1500 nm, and the fluorescence emission spectrum has a maximum value at 620 nm to 1700 nm, and

modifying the dye such that solubility is 10 uM or more in a 10 mM 4-(2-hydroxyethyl)-1-piperazineethanesulfonic acid (HEPES) solution with a pH of 7.4.

Further, in an exemplary embodiment of the present invention, the contrast agent further includes a ligand in order to attach the dye to tumor cells or tumor tissues, and the ligand may be selected such that the net charge of the contrast agent is +4, +3, +2, +1, 0, −1, −2, −3, or −4.

The contrast agent according to the present invention is characterized in that a charge of a conjugate of the dye and the ligand is balanced to promote renal excretion of the remaining material after tumor targeting.

As a ligand suitable for the contrast agent according to the present invention, TL indicated in the compound of Chemical Formula 1 or 2 may be used, and specifically, it is possible to use Arg-Gly-Asp (RGD) of the following Chemical Formula 6, cRGDyK of Chemical Formula 7, cRGDfK of Chemical Formula 8, folate of Chemical Formula 9, and the like.

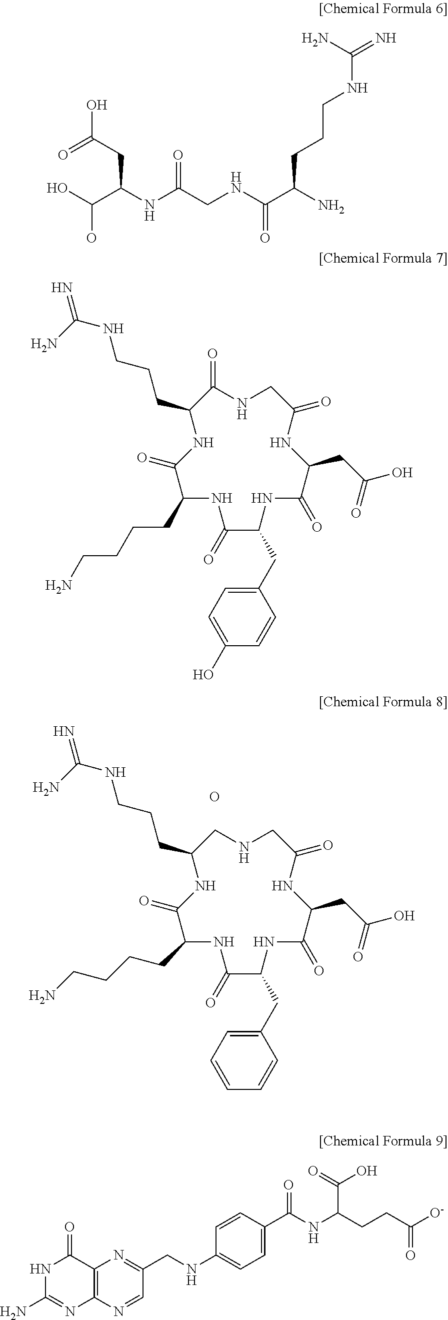

The ligand is for attaching the dye to cells or tissues, and a peptide including RGD binds tightly to endothelial cells activated during tumor angiogenesis and to αvb3-integrins or folate receptors which mediate extracellular matrix proteins, thereby enabling tumor-specific diagnosis.

Hereinafter, the present invention will be described in more detail through the Examples. These examples are merely for describing the present invention, and it should be obvious to those skilled in the art that the scope of the present invention is not limited to these examples.

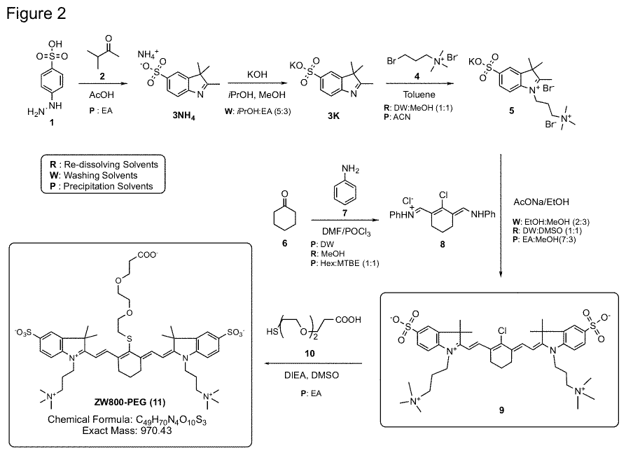

The following Examples will be described with reference to FIG. 2. The entire production method of Example 1 below is illustrated in FIG. 2, and the compounds produced in Examples 2 to 24 are illustrated in FIG. 1.

### Example 1: Production of Compound Contrast Agent (11) of Chemical Formula 1 or 2 (10 g Scale) (See FIG. 2)

### Example 1-1: Production of ammonium 2,3,3-trimethyl-3H-indole-5-sulfonate (3NH₄)

A 1 L round-bottom flask equipped with an oil bath, a stirrer bar, a reflux condenser, a rubber septum, and a nitrogen injector was prepared. And then, after nitrogen was injected, 4-hydrazinobenzenesulfonic acid 1 (60 g, 319 mmol) was weighed and put into the reaction flask. Glacial acetic acid (AcOH) (300 ml) was measured in a graduated cylinder and added to the reaction flask. Then, 3-methyl-2-butanone (2) (45 mL, 418 mmol) was added thereto by a 50 mL syringe. The flask was stirred under a nitrogen atmosphere for 18 hours while being heated to a reflux temperature (118±3° C.). After 18 hours, the heating was stopped, the oil bath was removed, and the flask was cooled to room temperature. In this case, the solution in the flask was dark red.

A 3 L flask was prepared on the stirrer and 2 L of EA was added to the flask. The solution in the reaction flask was slowly poured into the 3 L flask, and a pink precipitate was produced. After the solution was vigorously stirred for at least 30 minutes, the pink solid was filtered using a Buchner funnel. Then, the precipitate was washed with 500 mL of EA. The filtered precipitate was dried in a vacuum desiccator for 24 hours (overnight). And then, a bright pink solid ammonium 2,3,3-trimethyl-3H-indole-5-sulfonate (3NH₄)(65 g, 80% yield) was obtained, and no additional purification process was performed.

### Example 1-2: Production of potassium 2,3,3-trimethyl-3H-indole-5-sulfonate (3K)

Potassium hydroxide (17 g, 300 mmol) was prepared in a 500 mL Erlenmeyer flask, the flask was closed with a rubber septum, and then nitrogen was injected. And then, 200 mL of anhydrous isopropanol was injected by a syringe. The resulting solution was stirred well, heat was applied to increase the dissolution rate, and an ultrasonic disperser was used.

A 1 L round-bottom flask was prepared (24/40 joint), and 500 mL of MeOH was added thereto. And then, 65 g of ammonium 2,3,3-trimethyl-3H-indole-5-sulfonate (3NH₄) (254 mmol) prepared in Example 1-1 was added to the MeOH. In this case, the solution was red. After it was confirmed that all solids were dissolved, the prepared potassium hydroxide solution was added drop-wise using a funnel to stir the 3NH₄ solution. In this case, a yellow precipitate was formed. After all the potassium hydroxide solution was added, the resulting solution was stirred for 1 hour, and the reaction was completed. The yellow solid was filtered and washed with 200 mL of isopropanol and then 300 ml of EA. The washed solid was collected in a 500 mL brown glass bottle and dried in a vacuum desiccator for 24 hours to obtain an orange solid potassium 2,3,3-trimethyl-3H-indole-5-sulfonate (3K) (55 g, 78% yield).

### Example 1-3: Production of 2,3,3-trimethyl-5-sulfo-1-[3-(trimethylammonio)propyl]-3H-indolium dibromide (5)

A 1 L round-bottom flask equipped with an oil bath, a stirrer bar, a reflux condenser, a rubber septum, and a nitrogen injector was prepared, and nitrogen was injected thereinto. Potassium 2,3,3-trimethyl-3H-indole-5-sulfonate (3K) (23 g, 83 mmol) was weighed and put into the reaction flask under a nitrogen atmosphere. And then, (3-bromopropyl)trimethylammonium bromide (4) (25 g, 96 mmol) was added to the reaction flask, and 350 ml of toluene was injected thereinto by a syringe. The resulting solution was heated under reflux while being stirred. The reactants were not dissolved, and the reaction occurred in a static state. The reaction mixture was refluxed at 70° C. for 48 hours while being stirred under a nitrogen atmosphere. After 48 hours, the oil bath was removed, and the flask was cooled at room temperature. When the temperature reached room temperature, the stirring was stopped. Then, the solvent was discarded as much as possible while leaving the solid in the flask. 100 ml of MeOH was added thereto, and the resulting solution was stirred for 30 minutes to 1 hour until all solids were dissolved. The solid was filtered, and washed using 100 ml of CAN. And then, the filtered solid was re-dissolved in water (HPLC, 100 ml) and methanol (100 ml).

A 2 L flask was prepared on the stirrer and 2 L of ACN was added to the flask. Then, the mixed solution was added drop-wise to the CAN using a dropping funnel. A produced precipitate was transferred to a 250 ml brown glass bottle and dried under vacuum for 12 hours or more to obtain a pink solid 2,3,3-trimethyl-5-sulfo-1-[3-(trimethylammonio)propyl]-3H-indolium dibromide (16 g, 30 mmol, 36% yield), and no additional purification process was performed.

### Example 1-4: Production of 2-[4-chloro-7-[3,3-dimethyl-1-(3-(trimethylammonio)propyl] indolin-2-ylidene]-3,5-(propane-1,3-diyl)-1,3,5-heptatrien-1-yl]-3,3-dimethyl-1-[(3-(trimethylammonio)propyl]-3H-indolium dibromide (9)

A 300 mL round-bottom flask equipped with an oil bath, a stirrer bar, a reflux condenser, a rubber septum, and a nitrogen injector was prepared, and nitrogen was injected thereinto. N-{[(3E)-3-(anilinomethylene)-2-chlorocyclohex-1-en-1-yl]methylene}benzen-aminium chloride (8) (13.9 mmol) was added to the flask. And then, after 2,3,3-trimethyl-5-sulfo-1-(trimethylammonio)propyl]-3H-indolium dibromide (5) (15 g, 27.9 mmol) was added thereto, sodium acetate (3.42 g, 41.7 mmol) was added to the reaction flask. 200 mL of anhydrous ethanol was added thereto by a cannula or syringe, and the resulting solution was refluxed while being stirred at 85±2° C. for 6 hours. The solution turned brown and a brown precipitate was produced. The temperature was cooled to room temperature, and the precipitate was filtered. Using a spatula, the solid filtered by 200 mL of ethanol was washed while being gently stirred, and the solid was washed again with 300 mL of methanol.

The green-brown precipitate was collected in a brown glass bottle and dried under vacuum. And then, the dried solid was dissolved in 400 mL of a DMSO/water (1:1) mixed solvent, and 6 L of 70:30 EA/MeOH was slowly poured to cause precipitation. The precipitate was filtered under vacuum and dried for 24 hours (overnight) to obtain a brown solid 2-[4-chloro-7-[3,3-dimethyl-1-(3-(trimethylammonio)propyl]indolin-2-ylidene]-3,5-(propane-1,3-diyl)-1,3,5-heptatrien-1-yl]-3,3-dimethyl-1-[(3-(trimethylammonio)propyl]-3H-indolium dibromide (9) (10.2 g, 90.2% yield), and no additional purification process was performed.

### Example 1-5: Production of 3-(2-(2-(((E)-2-(3,3-dimethyl 1-5-sulfonato-1-(3-(trimethylammonio)propyl)-3H-indol-1-ium-2-yl)vinyl)-6-(2-((E)-3,3-dimethyl 1-5-sulfonato-1-(3-(trimethylammonio)propyl)indolin-2-ylidene)ethylidene)cyclohex-1-en-1-yl)thio)ethoxy)ethoxy)propanoate (11)

A 1 L round-bottom flask dried in an oven at 100° C. for 12 hours was prepared. Compound 9 (10 g, 12.3 mmol) produced in Example 1-4 was added to the flask along with a stirrer bar. And then, 600 mL of DMSO was added to a 1000 mL flask, and 4 equivalents of 3-(2-mercaptoethoxy)ethoxy)propanoic acid (10) (10.3 g, 49.2 mmol) was added thereto. The reaction flask was heated in an oil bath at 60° C. for 1 hour.

Then, the mixture of the heated reaction flask was cooled to room temperature, and added drop-wise to a flask prepared by adding 4 L of EA. And then, the flask was dried under vacuum to obtain 3-(2-(2-(((E)-2-((E)-2-(3,3-dimethyl-5-sulfonato-1-(3-(trimethylammonio)propyl)-3H-indol-1-ium-2-yl)vinyl)-6-(2-((E)-3,3-dimethyl-5-sulfonato-1-(3-(trimethylammonio)propyl)indolin-2-ylidene)ethylidene)cyclohex-1-en-1-yl)thio)ethoxy)ethoxy)propanoate (11) (10.2 g, 75% yield), and no additional purification process was performed.

### Examples 2 to 24: Production of Compound Contrast Agent (11) of Chemical Formula 1 or 2 (10 g Scale) (See FIG. 1)

Among the compounds of Chemical Formula 1 or 2 according to the present invention used as the contrast agent, representative compounds are selected from the group of the following Table 1.

TABLE 1

| Example | | R¹, R², R³, R⁴, Z, L |
|---|---|---|
| Chemical | 1 | R¹ = R² = COOH, R³ = R⁴ = N(CH₃)₃, Z = (CH₃)_{2,} L = S |
| Formula 1 | 2 | R¹ = R² = COOH, R³ = R⁴ = N(CH₃)₃, Z = (CH₃)_{2,} L = Se |
| | 3 | R¹ = R² = COOH, R³ = R⁴ = N(CH₃)₃, Z = (CH₃)_{2,} L = O |
| | 4 | R¹ = R² = COOH, R³ = R⁴ = N(CH₃)₃, Z = = (CH₃)_{2,} L = CH₂ |
| | 5 | R¹ = R² = SO₃H, R³ = R⁴ = N(CH₃)₃, Z = (CH₃)_{2,} L = S |
| | 6 | R¹ = R² = SO₃H, R³ = R⁴ = N(CH₃)₃, Z = (CH₃)_{2,} L = Se |
| | 7 | R¹ = R² = SO₃H, R³ = R⁴ = N(CH₃)₃, Z = (CH₃)_{2,} L = O |
| | 8 | R¹ = R² = SO₃H, R³ = R⁴ = N(CH₃)₃, Z = (CH₃)_{2,} L = CH₂ |
| | 9 | R¹ = R² = PO₃H₂, R³ = R⁴ = N(CH₃)₃, Z = (CH₃)_{2,} L = S |
| | 10 | R¹ = R² = PO₃H₂, R³ = R⁴ = N(CH₃)₃, Z = (CH₃)_{2,} L = Se |
| | 11 | R¹ = R² = PO₃H₂, R³ = R⁴ = N(CH₃)₃, Z = (CH₃)_{2,} L = O |
| | 12 | R¹ = R² = PO₃H₂, R³ = R⁴ = N(CH₃)₃, Z = (CH₃)_{2,} L = CH₂ |
| Chemical | 13 | R¹ = R² = COOH, R³ = R⁴ = N(CH₃)₃, Z = (CH₃)_{2,} L = S |
| Formula 2 | 14 | R¹ = R² = COOH, R³ = R⁴ = N(CH₃)₃, Z = (CH₃)_{2,} L = Se |
| | 15 | R¹ = R² = COOH, R³ = R⁴ = N(CH₃)₃, Z = (CH₃)_{2,} L = O |
| | 16 | R¹ = R² = COOH, R³ = R⁴ = N(CH₃)₃, Z = (CH₃)_{2,} L = CH₂ |
| | 17 | R¹ = R² = SO₃H, R³ = R⁴ = N(CH₃)₃, Z = (CH₃)_{2,} L = S |
| | 18 | R¹ = R² = SO₃H, R³ = R⁴ = N(CH₃)₃, Z = (CH₃)_{2,} L = Se |
| | 19 | R¹ = R² = SO₃H, R³ = R⁴ = N(CH₃)₃, Z = (CH₃)_{2,} L = O |
| | 20 | R¹ = R² = SO₃H, R³ = R⁴ = N(CH₃)₃, Z = (CH₃)_{2,} L = CH₂ |
| | 21 | R¹ = R² = PO₃H₂, R³ = R⁴ = N(CH₃)₃, Z = (CH₃)_{2,} L = S |
| | 22 | R¹ = R² = PO₃H₂, R³ = R⁴ = N(CH₃)₃, Z = (CH₃)_{2,} L = Se |
| | 23 | R¹ = R² = PO₃H₂, R³ = R⁴ = N(CH₃)₃, Z = (CH₃)_{2,} L = O |
| | 24 | R¹ = R² = PO₃H₂, R³ = R⁴ = N(CH₃)₃, Z = (CH₃)_{2,} L = CH₂ |

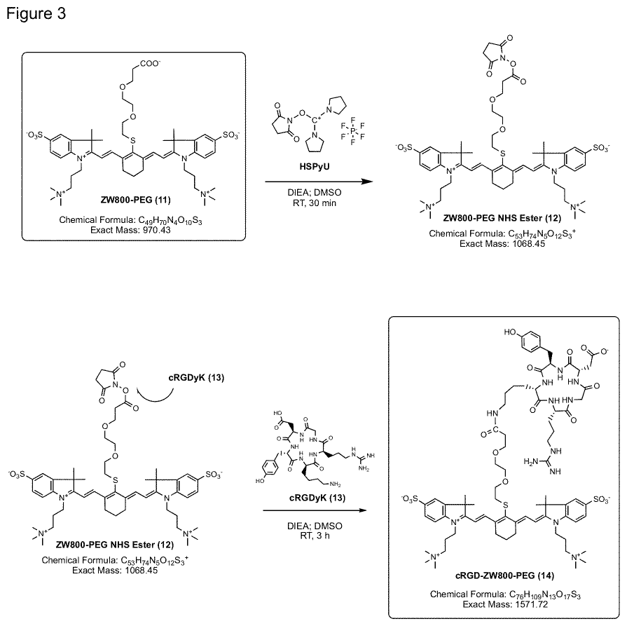

### Example 25: Production of Ligand-Bound Contrast Agent (1 g Scale) (See FIG. 3)

A ligand cRGDyK was bound to the contrast agent produced in Example 1. Furthermore, referring to such a production method, the ligands were respectively bound to the contrast agents produced in Examples 2 to 24.

### Example 25-1: Production of ZW800-PEG NHS Ester (12)

After 971 mg (1 μmol) of Compound 11 (ZW800-PEG) produced in Example 1-5 was dissolved in anhydrous DMSO, 822 mg (2 μmol) of HSPyU was added thereto, and the solution was mixed for 5 minutes. And then, 20 μL of DIEA (about 2 equiv) was added thereto, and the pH of the solution was confirmed. DIEA was added to adjust the reaction pH to 10 (in this case, excessive addition of DIEA may destroy the backbone of the dye). After the solution was mixed for 30 minutes, HPLC was checked to terminate the reaction (>95%). After the reaction was terminated, the solution was poured into 500 ml of ethyl acetate, and then washed with a mixed solution of acetone/ethanol (500 mL, 1:1 v/v). After washing, the precipitate was collected separately and dried for 24 hours to obtain a solid dye (12) (96.4% yield).

### Example 25-2: Production of cRGD-ZW800-PEG (14)

930 mg of cRGDyK (13) (1.5 μmol) was dissolved in a 1 L reaction flask including 500 ml of PBS (pH 8.0). And then, a solution obtained by dissolving 1,068 mg (1 μmol) of the compound (12) produced in Example 25-1 in 200 ml of DMSO was added thereto. After the solution was mixed for 3 hours, HPLC was checked to terminate the reaction (>95%). And then, after the reaction solution was poured into 500 mL of ethyl acetate, the resulting mixture was washed with a mixed solution of acetone/ethanol (500 ml, 1:1 v/v). After washing, the precipitate was collected separately and dried for 24 hours to obtain a solid dye (14) (75.8% yield).

### Experimental Example 1: Measurement of Tumor Signal-to-Background Ratio

The tumor signal-to-background ratio is measured by a fluorescence imaging device, a target tissue (for example, cancer) is compared with a comparative tissue (for example, muscle), and the ratio is shown as a number.

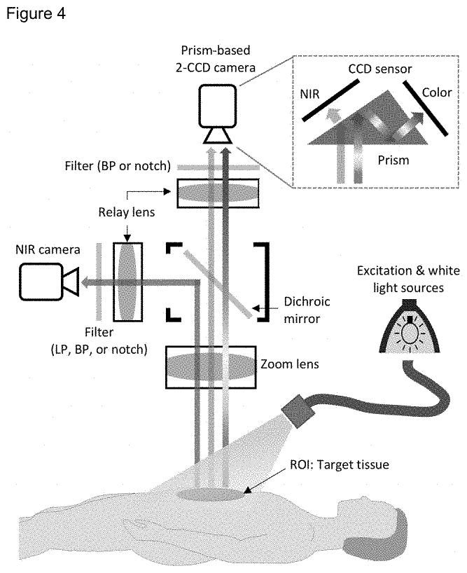

Table 2 shows a comparison of the types of currently commercialized real-time image equipment and the main performance thereof, and the image measurement method is illustrated in FIG. 4. In addition to the equipment, the signal-to-background ratio may also be measured using an image processing software after obtaining the image.

TABLE 2

| Feature | PDE | Fluobeam | SPY | Solaris | Lab-Flare | FIAT-L |
|---|---|---|---|---|---|---|
| Manufacturer | Hamamatsu | Fluoptics | Novadaq | Perkin | Curadel | Nawoo Vision |
| | Photonics | | Tech. | Elmer | | |
| Country | Japan | France | Canada | USA | USA | South Korea |
| Fluorescence | CCD | CCD | CCD | sCMOS | CCD | CCD |
| camera | | | | | | |
| Sensitivity (nM) | 15 | 5 | 5 | 10 | 1 | 1 |
| Excitation (nm) | 760 | 690 or 780 | 820 | 465, 660, | 665 or 760 | 660 or 760 |
| | | | | 735 or 790 | | |
| Excitation light | LED | LD | LD | LED | LD | LD |
| source | | | | | | |
| Fluence rate | 4 | 6 | 31 | — | 4/14 | 4/10 |
| (mW/cm²) | | | | | | |
| Dynamic range | 8 | 12 | 8 | 12 | 10 | 12 |
| (bits) | | | | | | |
| Field-of-view | 10 × 6.7 | 20 × 14 | 19 × 14 | 10 × 10 | 25 × 25 | 15 × 15 |
| (cm) | | | | | | |
| Working | 20 | 15-25 | 30 | 75 | 30-45 | 10-50 |
| distance (cm) | | | | | | |
| Zoom | No. | No. | No. | Yes | Yes | Yes |
| Reference | hamamatsu.com | fluoptics.com | novadaq.com | perkinelmer.com | curadel.com | nawoovision.com |

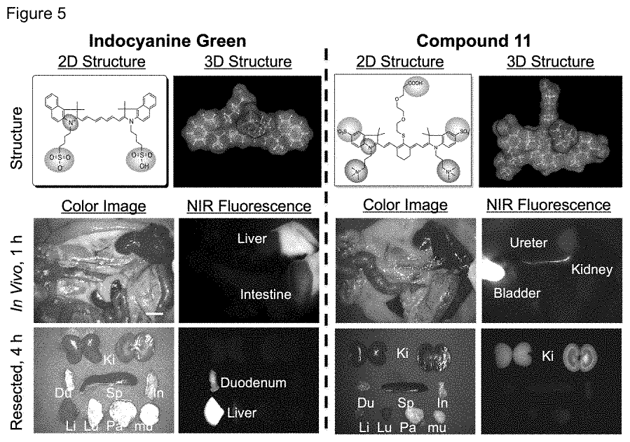

Referring to FIG. 5, the structures of FDA-approved indocyanine Green and Compound 11 are compared, and below that, the biodistribution maps of the two contrast agents 1 hour after the contrast agents were injected into SD rats and the signal-to-background ratios of the excised organs after 4 hours were compared.

The tumor signal-to-background ratios for the compounds of Examples 1-24 according to the present invention are shown in Table 3 below.

TABLE 3

| Example | Signal-to-background ratio |
|---|---|
| 1 | 2.4 |
| 2 | 2.6 |
| 3 | 1.8 |
| 4 | 2.1 |
| 5 | 2.9 |
| 6 | 2.4 |
| 7 | 2.1 |
| 8 | 2.3 |
| 9 | 2.1 |
| 10 | 2.2 |
| 11 | 1.6 |
| 12 | 2.0 |
| 13 | 3.1 |
| 14 | 3.2 |
| 15 | 2.4 |
| 16 | 2.9 |
| 17 | 3.9 |
| 18 | 3.2 |
| 19 | 2.9 |
| 20 | 3.4 |
| 21 | 3.0 |
| 22 | 3.2 |
| 23 | 2.6 |
| 24 | 3.1 |

Therefore, referring to Table 3, it could be seen that the contrast agent according to the present invention has a high tumor signal relative to the background signal, and thus has selective specificity for tumor tissues and cells.

Although a specific part of the present invention has been described in detail, it will be obvious to a person with ordinary skill in the art to which the present invention pertains that such a specific description is just a preferred embodiment and the scope of the present invention is not limited thereto. A person with ordinary skill in the art to which the present invention pertains will be able to make various applications and modifications within the scope of the present invention based on the aforementioned contents.

Accordingly, the substantial scope of the present invention will be defined by the appended claims and equivalents thereof.

### INDUSTRIAL APPLICABILITY

The fluorescence contrast agent according to the present invention can be applied to a disease imaging diagnostic field using near infrared fluorescence such as X-ray imaging and MRI.

1. A renal excretion-type fluorescence contrast agent for improving a tumor signal-to-background ratio, which is a contrast agent for imaging tumor tissues or tumor cells,
wherein the contrast agent comprises a compound which emits fluorescence such that a tumor signal-to-background ratio is detected at 1.1 or higher,
the compound is a compound of Chemical Formula 1 or 2, and
the contrast agent increases renal excretion of the materials remaining in vivo after tumor targeting: 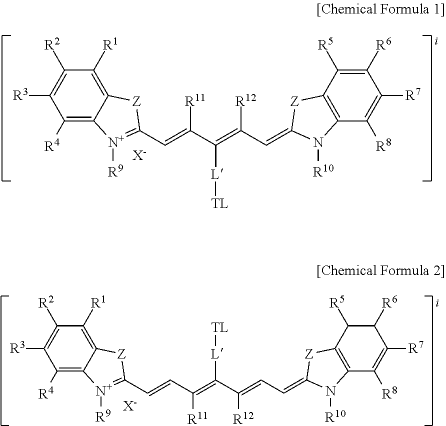
in the formulae,
R¹ to R¹² are each independently hydrogen, (Y)ₘSO₃⁻, (Y)ₘCOO⁻, (Y)ₘPO₃H⁻, (Y)ₘNH₂(CH₃)⁺, (Y)ₘNH(CH₃)₂⁺, (Y)ₘN(CH₃)₃⁺, a C₁-C₄ alkyl group, a C₁-C₄ alkoxy group, or a C₂-C₁₂ alkenyl group,
R¹¹ and R¹² are optionally linked to each other to form an aliphatic or aromatic ring,
Y is CH₂ or CH₂CH₂O,
m is an integer from 0 to 9,
L′ is a structure linked to CH₂, hydrogen (H), oxygen (O), nitrogen (NH), sulfur (S), or selenide (Se),
TL is a targeting ligand,
X is F, Cl, Br, or I,
Z is S, Se, O, CH═CH, or C(CH₃)₂, and
i is +4, +3, +2, +1, 0, −1, −2, −3, or −4.

2. The renal excretion-type fluorescence contrast agent of claim 1, wherein L′ comprises a PEG linker represented by a compound of the following Chemical Formula 3: 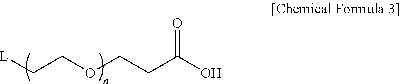
in the formula,
L is S, Se, O, or CH₂, and
n is an integer from 0 to 114.

3. The renal excretion-type fluorescence contrast agent of claim 2, wherein L′ is -L-PEG-(CH₂)₂CONH—.

4. The renal excretion-type fluorescence contrast agent of claim 1, wherein the compound of Chemical Formula 1 is used when the contrast agent absorbs light in a region of 700 nm, and the compound of Chemical Formula 2 is used when the contrast agent absorbs light in a region of 800 nm.

5. The renal excretion-type fluorescence contrast agent of claim 1, wherein the contrast agent further comprises a compound of the following Chemical Formula 4 or 5: 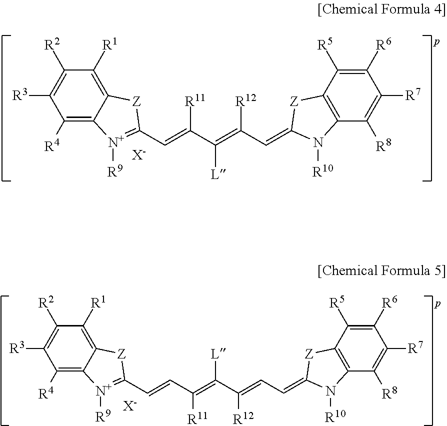
in the formulae,
R¹ to R¹² are each independently hydrogen, a C₁-C₄ alkyl group, a C₁-C₄ alkoxy group, or a C₂-C₁₂ alkenyl group,
L″ is hydrogen (H), fluorine (F), chlorine (Cl), bromine (Br), or benzene,
X is F, Cl, Br, or I,
Z is S, Se, O, CH═CH, or C(CH₃)₂, and
p is an integer from −6 to +6.

6. An imaging method using a fluorescence agent for increasing renal excretion, the method comprising: (a) attaching a phosphor represented by a compound of the following Chemical Formula 1 or 2, or a contrast agent comprising the same to tumor tissues or cells;
(b) emitting fluorescence from the tumor tissues or cells by causing the phosphor or contrast agent exposed to an excitation light source to produce fluorescence;
(c) increasing renal excretion of the remaining materials in the body after tumor targeting by emitting fluorescence; and
(d) imaging the tumor tissues or cells by observing a fluorescence signal emitted from the tumor tissues or cells at a signal-to-background ratio of 1.1 or higher: 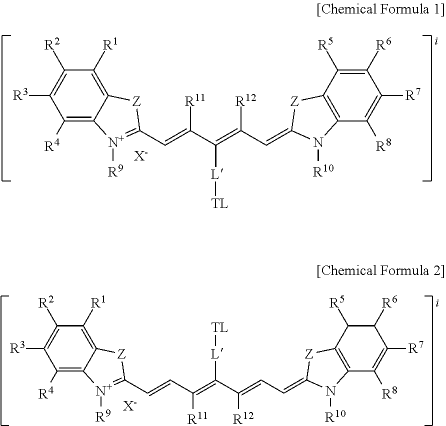
in the formulae,
R¹ to R¹² are each independently hydrogen, (Y)ₘSO₃⁻, (Y)ₘCOO⁻, (Y)ₘPO₃H⁻, (Y)ₘNH₂(CH₃)⁺, (Y)ₘNH(CH₃)₂⁺, (Y)ₘN(CH₃)₃⁺, a C₁-C₄ alkyl group, a C₁-C₄ alkoxy group, or a C₂-C₁₂ alkenyl group,
R¹¹ and R¹² may be linked to each other to form an aliphatic or aromatic ring,
Y is CH₂ or CH₂CH₂O,
m is an integer from 0 to 150,
L′ is a structure linked to CH₂, hydrogen (H), oxygen (O), nitrogen (N), sulfur (S), or selenide (Se),
TL is a targeting ligand,
X is F, Cl, Br, or I,
Z is S, Se, O, CH═CH, or C(CH₃)₂, and
i is +4, +4, +2, +1, 0, −1, −2, −3, or −4.

7. The method of claim 6, wherein L′ comprises a PEG linker represented by a compound of the following Chemical Formula 3: 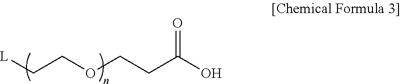
in the formula,
L is S, Se, O, or CH₂, and
n is an integer from 0 to 114.

8. The method of claim 7, wherein L′ is -L-PEG-(CH₂)₂CONH—.

9. The method of claim 6, where the contrast agent is produced by:
selecting a dye whose absorption spectrum has a maximum value at 600 nm to 1500 nm, and the fluorescence emission spectrum has a maximum value at 620 nm to 1700 nm, and
modifying the dye such that solubility is 10 uM or more in a 10 mM 4-(2-hydroxyethyl)-1-piperazineethanesulfonic acid (HEPES) solution with a pH of 7.4.

10. The method of claim 6, where the contrast agent further comprises a ligand for attaching the dye to tumor cells or tumor tissues,
wherein the ligand is selected such that the net charge of the contrast agent is +4, +3, +2, +1, 0, −1, −2, −3, or −4, and
a charge of a conjugate of the dye and the ligand is balanced to promote renal excretion of the remaining material after tumor targeting.
